(19) United States
(12) Patent Application Publication
(10) Pub. No.: US 2020/0185097 A1
(43) Pub. Date: Jun. 11, 2020

(54) GUIDING USER MOTION FOR PHYSIOTHERAPY IN VIRTUAL OR AUGMENTED REALITY

(71) Applicant: XR Health IL LTD, Tel Aviv (IL)

(72) Inventors: Eran Orr, Brookline, MA (US); Sagie Grunhaus, Tel Aviv (IL)

(21) Appl. No.: 16/791,665

(22) Filed: Feb. 14, 2020

Related U.S. Application Data
(63) Continuation of application No. PCT/US18/00328, filed on Aug. 17, 2018.
(60) Provisional application No. 62/546,818, filed on Aug. 17, 2017.

(51) Int. Cl. G16H 40/67; G16H 20/30; G16H 10/60; G06F 3/01; G06T 19/00

(52) U.S. Cl. CPC G16H 40/67; G16H 20/30; G06T 2210/41; G06F 3/012; G06T 19/006; G16H 10/60

(57) ABSTRACT

Guidance of user motion for physiotherapy in a virtual reality or augmented reality environment is provided. In various embodiments, an object is displayed to a user within a virtual environment. The user is directed to track the object within the virtual environment with a body part. The object is moved within the virtual environment to induce motion of the body part in compliance with a predetermined rehabilitation protocol. The position of the body part is determined and the position is sent over a network to a remote server. Compliance with the predetermined rehabilitation protocol is determined at the remote server. An electronic health record includes the predetermined rehabilitation protocol and may be stored in a remote database.

### CROSS-REFERENCE TO RELATED APPLICATIONS

This application claims the benefit of U.S. Provisional Application No. 62/546,818 filed Aug. 17, 2017, which is hereby incorporated by reference in its entirety.

### BACKGROUND

Embodiments of the present disclosure relate to physical therapy using virtual or augmented reality, and more specifically, to guiding user motion for physiotherapy in virtual reality (VR) or augmented reality (AR) environments.

### BRIEF SUMMARY

According to embodiments of the present disclosure, methods of, systems for, and computer program products for guiding user motion are provided. In various embodiments, a method of treatment for guiding a patient through a rehabilitation protocol are provided. In various embodiments, an object is displayed to a user within a virtual environment. The user is directed to track the object within the virtual environment with a body part. The object is moved within the virtual environment to induce motion of the body part in compliance with a predetermined rehabilitation protocol. The position of the body part is determined. The position of the body part is received at a remote server. Compliance with the predetermined rehabilitation protocol may be determined at the remote server.

In various embodiments, a system is provided including a virtual reality display adapted to display a virtual environment to a user and a computing node comprising a computer readable storage medium having program instructions embodied therewith, the program instructions executable by a processor of the computing node to cause the processor to perform a method. In performing the method, an object is displayed to a user within a virtual environment. The user is directed to track the object within the virtual environment with a body part. The object is moved within the virtual environment to induce motion of the body part in compliance with a predetermined rehabilitation protocol. The position of the body part is determined. The position of the body part is received at a remote server. Compliance with the predetermined rehabilitation protocol may be determined at the remote server.

In various embodiments, computer program products for guiding user motion are provided, the computer program product comprising a computer readable storage medium having program instructions embodied therewith, the program instructions executable by a processor to cause the processor to perform a method. In performing the method, an object is displayed to a user within a virtual environment. The user is directed to track the object within the virtual environment with a body part. The object is moved within the virtual environment to induce motion of the body part in compliance with a predetermined rehabilitation protocol. The position of the body part is determined. The position of the body part is received at a remote server. Compliance with the predetermined rehabilitation protocol is determined at the remote server

In various embodiments, a plurality of positions of the body part is determined. In various embodiments, determining compliance includes comparing the plurality of positions of the body part with a plurality of predetermined positions, determining a compliance factor based on the comparing, and determining whether the compliance factor is above a predetermined threshold. In various embodiments, the plurality of predetermined positions represent positions along a three-dimensional path corresponding to the rehabilitation protocol. In various embodiments, comparing includes determining a difference between the plurality of positions of the body part and the plurality of predetermined positions. In various embodiments, the difference includes an absolute difference. In various embodiments, determining compliance comprises determining compliance with an electronic health record. In various embodiments, the electronic health record contains the predetermined rehabilitation protocol. In various embodiments, the electronic health record is stored in a remote database. In various embodiments, in performing the method, a result of the determining compliance is logged into the electronic health record. In various embodiments, in performing the method, the user is directed to assume a predetermined posture before the user is directed to track the object. In various embodiments, in performing the method, whether the user has assumed the predetermined posture is determined. In various embodiments, in performing the method, the user is not directed to track the object until the predetermined posture has been assumed. In various embodiments, determining the position of the body part includes determining a three-dimensional coordinate of a sensor attached to the body part.

### BRIEF DESCRIPTION OF THE SEVERAL VIEWS OF THE DRAWINGS

FIG. 1 illustrates an exemplary virtual reality headset according to embodiments of the present disclosure.

FIGS. 2A-2F illustrate exemplary user motion according to embodiments of the present disclosure.

FIGS. 3A-3C illustrate exemplary user motion according to embodiments of the present disclosure.

FIGS. 4A-4H illustrate exemplary user motion according to embodiments of the present disclosure.

FIGS. 5A-5D illustrate exemplary user motion according to embodiments of the present disclosure.

FIG. 6 illustrates a method of guiding user motion according to embodiments of the present disclosure.

FIG. 7 depicts a computing node according to an embodiment of the present invention.

### DETAILED DESCRIPTION

Physical therapy attempts to address the illnesses or injuries that limit a person's abilities to move and perform functional activities in their daily lives. Physical therapy may be prescribed to address a variety of pain and mobility issues across various regions of the body. In general, a program of physical therapy is based on an individual's history and the results of a physical examination to arrive at a diagnosis. A given physical therapy program may integrate assistance with specific exercises, manual therapy and manipulation, mechanical devices such as traction, education, physical agents such as heat, cold, electricity, sound waves, radiation, assistive devices, prostheses, orthoses and other interventions. Physical therapy may also be prescribed as a preventative measure to prevent the loss of mobility before it occurs by developing fitness and wellness-oriented programs for healthier and more active lifestyles. This may include providing therapeutic treatment where movement and function are threatened by aging, injury, disease or environmental factors.

As an example, individuals suffer from neck pain or need to perform neck exercises for various reasons. For example, people who have been involved in a motor vehicle accident or have suffered an injury while playing contact sports are prone to develop a whiplash associated disorder (WAD), a condition resulting from cervical acceleration-deceleration (CAD). It will be appreciated that this is just one of many potential injuries that may result in neck injury or pain necessitating rehabilitation.

The majority of people who suffer from non-specific neck pain (NSNP) may have experienced symptoms associated with WAD or have an undiagnosed cervical herniated disc. For this population, the recommended treatment regimen often includes a variety of exercises promoting neck movement and other functional activity training, leading to improved rehabilitation.

Poor adherence to treatment can have negative effects on outcomes and healthcare cost, irrespective of the region of the body affected. Poor treatment adherence is associated with low levels of physical activity at baseline or in previous weeks, low in-treatment adherence with exercise, low self-efficacy, depression, anxiety, helplessness, poor social support/activity, greater perceived number of barriers to exercise and increased pain levels during exercise. Studies have shown that about 14% of physiotherapy patients do not return for follow-up outpatient appointments. Other studies have suggested that overall non-adherence with treatment and exercise performance may be as high as 70%. Patients that suffer from chronic or other long-term conditions (such as those associated with WAD or NSNP) are even less inclined to perform recommended home training.

Adherent patients generally have better treatment outcomes than non-adherent patients. However, although many physical therapy exercises may be carried out in the comfort of one's home, patients cite the monotony of exercises and associated pain as contributing to non-adherence.

Irrespective of adherence, home training has several limitations. With no direct guidance from the clinician, the patient has no immediate feedback to confirm correct performance of required exercises. Lack of such guidance and supervision often leads to even lower adherence. As a result, the pain of an initial sensed condition may persist or even worsen—leading to other required medical interventions that could have been prevented, thus also increasing associated costs of the initial condition.

Accordingly, there is a need for devices, systems, and methods that facilitate comprehensive performance and compliance with physical therapy and therapeutic exercise regimens.

According to various embodiments of the present disclosure, various devices, systems, and methods are provided to facilitate therapy and physical training assisted by virtual or augmented reality environments.

It will be appreciated that a variety of virtual and augmented reality devices are known in the art. For example, various head-mounted displays providing either immersive video or video overlays are provided by various vendors. Some such devices integrate a smart phone within a headset, the smart phone providing computing and wireless communication resources for each virtual or augmented reality application. Some such devices connect via wired or wireless connection to an external computing node such as a personal computer. Yet other devices may include an integrated computing node, providing some or all of the computing and connectivity required for a given application.

Virtual or augmented reality displays may be coupled with a variety of motion sensors in order to track a user's motion within a virtual environment. Such motion tracking may be used to navigate within a virtual environment, to manipulate a user's avatar in the virtual environment, or to interact with other objects in the virtual environment. In some devices that integrate a smartphone, head tracking may be provided by sensors integrated in the smartphone, such as an orientation sensor, gyroscope, accelerometer, or geomagnetic field sensor. Sensors may be integrated in a headset, or may be held by a user, or attached to various body parts to provide detailed information on user positioning.

In the course of a program of rehabilitation, patients follow physical training protocols that guide the physical aspect of their recovery and define what physical motions and activities are required for treatment. Such protocols often include repetitive motions and activities designed to activate and facilitate movement of specific body parts. The patient may be guided to follow and repeat these motions and activities through the assistance of external equipment (e.g., weights or bands) that can control resistance and difficulty.

As discussed above, traditional protocol training often exhibits low adherence. In many cases, low adherence may be attributed to the repetitive, unengaging nature of such protocols. To address this boredom, a user may watch a television screen while doing the motions and activities or listen to music. However, even with this additional stimulus, the motions and activities themselves continue to be tedious.

To address this and other limitations of alternative approaches, the present disclosure enables following training protocols while immersed in a virtual or augmented reality environment. According to various embodiments, content such as videos, movies, or 3D objects are displayed to a patient. The movement of this content in the space around the patient is used to guide the motions and activities defined by the protocol. This level of immersion encourages better adherence than watching a stationary screen.

The systems, methods and computer program products of the disclosure generally guide a patient/user through a rehabilitation protocol in a VR/AR environment. The VR/AR environment may display an object to the patient/user. In various embodiments, the object may be, for example, a light, a target, or one or more balloons. In various embodiments, the one or more objects may be a part of a game. The VR/AR environment may direct the patient/user to track the object within the virtual environment with a body part, such as, for example, a hand or head. In various embodiments, a patient/user may be presented with a target and instructed to motion with a body part towards the target upon receiving an indication to do so. For example, the patient/user may be presented with a grid of unlit light bulbs and instructed to motion towards a lightbulb when it turns on. In various embodiments, the lightbulb may have one color (e.g., blue) for one body part (e.g., left hand) and another color (e.g., red) for another body part (e.g., right hand).

In various embodiments, the patient/user may be presented with one or more objects arranged in a particular orientation for a rehabilitation exercise. For example, the patient/user may be presented with a line of balloons arranged, for example, horizontally, diagonally, vertically, and/or circularly. In various embodiments, the patient/user may be instructed to make a motion with one or both hands, thereby cutting through the balloons to work through one or both shoulder's range of motion. In various embodiments, the balloons may be colored to indicate a particular body part. For example, blue balloons are associated with the left hand/shoulder and red balloons are associated with the right hand/shoulder.

In various embodiments, the VR/AR system may include a range-of-motion (ROM) assessment before the game begins to establish a baseline ROM of a particular body part. For example, the VR/AR environment may direct a patient/user through a series of motions to determine a maximum ROM that is comfortable for the patient/user. In various embodiments, the VR/AR system may adjust the game based on the patient's/user's maximum ROM. In various embodiments, the ROM assessment is performed before each session and may be tracked over time and/or logged into an electronic health record.

In various embodiments, the system may not direct the user to track the object in the VR/AR environment until the patient/user assumes a particular posture for the exercise. In various embodiments, the particular posture is required at the beginning of the exercise. In various embodiments, the VR/AR system may require the patient/user to assume the particular posture at any point throughout the exercise. In various embodiments, the VR/AR system may require the patient/user to maintain a specific posture throughout an exercise. Such a requirement may prevent the patient/user from injuring themselves during the exercise.

In various embodiments, the VR/AR system may provide an indication to the patient/user that they are deviating from the required posture. For example, the indication may be a colored light where green indicates compliance with the particular posture, yellow indicates a slight deviation from the required posture, and red means a large deviation from the required posture. In various embodiments, the VR/AR system may pause the exercise at any suitable point if the patient/user is not compliant with the required posture.

In various embodiments, the patient/user may be assigned a score based on compliance with the training/rehabilitation protocol. For example, the patient/user may receive points for motioning towards a lightbulb of one color (e.g., blue) with their left hand while not receiving any points for motioning towards the same lightbulb with their right hand (and vice versa). In another example, the patient/user may receive more points for motioning towards a lightbulb of one color (e.g., blue) with their left hand while receiving fewer points for motioning towards the same lightbulb with their right hand. In another example, the patient/user may receive points for popping each balloon in a single motion thereby receiving the most points when all balloons are popped in a single motion.

In various embodiments, the object may be moved within the virtual environment to induce motion of the body part. In various embodiments, the motion may be a predetermined motion that is a part of a rehabilitation protocol. For example, a target may be moved within the VR/AR environment to induce the patient/user to motion in a particular direction towards the target.

In various embodiments, the VR/AR system may determine the position of the body part and record the position over time. In various embodiments, as described in more detail above, one or more sensors may be attached to or otherwise associated with a body part to track a three-dimensional position and motion of the body part with six degrees of freedom. In various embodiments, the system may determine a plurality of positions of one or more body parts. The plurality of positions may correspond to points along a three-dimensional path taken by the body part.

In various embodiments, the three-dimensional path may be compared to a predetermined path, for example, defined in a rehabilitation protocol to compute the error between the path taken by the body part of the patient/user and the path defined in the predetermined rehabilitation protocol. In various embodiments, the comparison may be made by taking the difference between the points defining the three-dimensional path taken by the body part and the predetermined path defined in the rehabilitation protocol. In various embodiments, the difference may be an absolute difference. In various embodiments, the difference may be a difference between the square of the points defining the three-dimensional path taken by the body part and the square of the predetermined path defined in the rehabilitation protocol.

In various embodiments, a remote server may receive the position and/or the plurality of positions of the body part. In various embodiments, the remote server may determine, from the received position (and/or plurality of positions) if the patient/user is compliant with the rehabilitation protocol. The determination of compliance may correspond to a score the patient/user receives while playing a game presented by the VR/AR environment, as described above. In various embodiments, the compliance factor may be logged in an electronic health record that is stored in a remote database. In various embodiments, the remote database may be located at the same or different server as the remote server.

In various embodiments, the compliance factors computed for a particular patient/user may be compared across time to determine whether the patient/user is improving. In various embodiments, an increasing compliance factor may indicate that the patient is responding to the rehabilitation protocol in a positive manner (e.g., an injury is improving).

In various embodiments, the system may track the position and motion of one or more eyes of the patient/user using methods as are known in the art. Eye tracking may be implemented in various embodiments where position/motion data may provide an indication (sole or additional) of compliance with a rehabilitation protocol.

In various embodiments, the system may track the position and motion of the head. In various embodiments, the system may utilize sensors in a head-mounted display to determine the position and motion of the head with six degrees of freedom as described above. Head tracking may be implemented in various embodiments where position/motion data provide an indication (sole or additional) of compliance with a rehabilitation protocol. For example, head tracking may be implemented when using a rehabilitation protocol that includes neck exercises.

In various embodiments, for more nuanced exercises, one or more additional sensors may provide position/motion data of various body parts to obtain appropriate data to determine compliance for the particular exercise.

In various embodiments, the systems of the present disclosure may be utilized in a method of treatment. In particular, a treatment plan may be received from a remote server, such as a server having an electronic health record database. In various embodiments, the treatment plan may include a predetermined rehabilitation protocol to be followed by the patient. As the patient uses the VR/AR systems described herein to follow the treatment plan, compliance with the treatment plan may be monitored and logged with the electronic health record. If the patient is not complying with the treatment plan, in various embodiments, the system may send an indication (e.g., a message) to a healthcare provider seeking oversight for the particular patient.

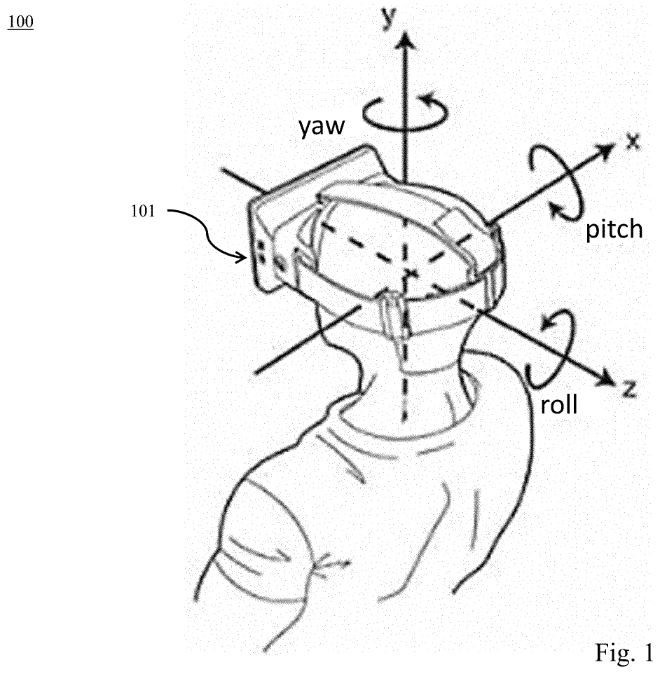

With reference now to FIG. 1, an exemplary virtual reality headset is illustrated according to embodiments of the present disclosure. In various embodiments, system 100 is used to collected data from motion sensors including hand sensors (not pictured), sensors included in headset 101, and additional sensors such as torso sensors or a stereo camera. In some embodiments, data from these sensors is collected at a rate of up to about 150 Hz. As pictured, data may be collected in six degrees of freedom: X—left/right; Y—up/down/height; Z—foreword/backward; P—pitch; R—roll; Y—yaw. As set out herein, this data may be used to track a user's overall motion and compliance with a predetermined exercise routine. Likewise, headset 101 may position various moving 2D or 3D objects to guide the user through physical training protocols.

In an exemplary 2D embodiment, the user may be prompted to follow a moving screen (e.g., playing videos) in the space around the user. In this way, the user is guided to continue performing repetitive motion in order to avoid losing sight of the screen, this directing the correct protocol motions.

In an exemplary 3D embodiment, a moving 3D character or scene moves in the space around the player, guiding the players motions and directing the correct protocol motions.

The approaches provided herein allow more complex motions than relying on conventional tensioners and weights. The VR or AR environment allows an essentially limitless range of moving elements around the user, which facilitation of the protocol motions needed. Likewise, the VR/AR environment provides an immersive environment, keeping players engaged and actively performing the necessary physical motions for the medical protocol.

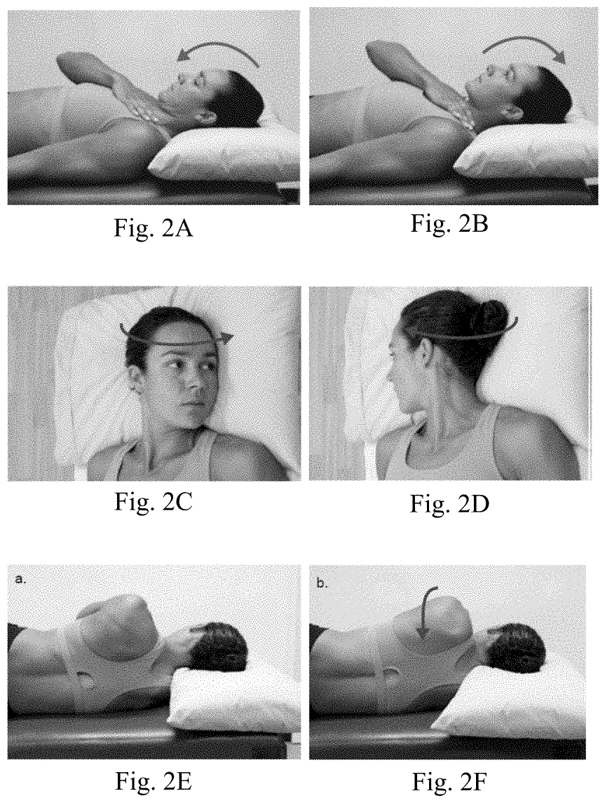

Referring now to FIGS. 2A-2F, 3A-3C, 4A-4F, and 5A-5D, various exemplary motions of a user's neck are illustrated. To facilitate these motions, in various embodiments, a moving 2D or 3D object is displayed through a VR or AR device to the user. This object moves around the user's space, guiding the performance of specific physical training protocols. The user, in order to follow the object and succeed in the training and/or rehabilitation, must physically perform the desired motions by following the object's movement in space. It will be appreciated that although the present example is given in terms of neck motions, tracking of the virtual object may be based on the motion of different body parts, depending on the training protocol performed. For example, a handheld sensor may be tracked, and the user prompted to move their arm to remain pointing at a virtual object. In various embodiments, other body parts may be tracked, such as, for example, one or both legs, one or both feet, one or both hands, one or both arms, and/or a user's head.

The following exercises can be used by practitioners when providing primary care (e.g., rehabilitation) to people, such as those suffering from WAD. The exercises are designed to restore the movement and muscle control around the neck and to reduce unnecessary postural strain and muscle pain. For each exercise, the patient/user may be instructed move smoothly and slowly, without sudden jerks; the key is precision and control. The patient/user may be instructed to keep their mouth and jaw relaxed; keep their lips together, teeth slightly apart and with the tongue resting on the roof of the mouth. The patient/user may be instructed to gently hold their shoulders back and down so that they are relaxed while they are performing all exercises. A posture correction exercise may be used to correct posture, as explained in more detail below. In movement exercises, the patient/user may be instructed to try to move the same distance to each side. If one side is stiffer, the patient/user may be instructed to move gently into the stiffness. The patient/user may be instructed to move to that direction a little more often. In the event the patient/user experiences some discomfort, may be instructed that exercises should not cause severe pain and, thus, should stop the exercise.

In various embodiments, the systems and methods described herein may not begin the rehabilitation session until the user has assumed a specific predetermined starting posture. This may improve the results of the rehabilitation exercises and/or improve compliance with the predetermined rehabilitation protocol.

FIGS. 2A-2F illustrate exemplary user motion according to embodiments of the present disclosure. In particular, FIGS. 2A-2F illustrate various neck exercises performed while laying down that may be utilized in various embodiments of the systems described herein.

FIGS. 2A-2B illustrate a chin nod exercise. The user may be instructed to gently and slowly nod their head forward as if to say ‘yes’. The user may be instructed to stop the nodding action just before they feel the front neck muscles hardening. The user may be instructed to hold the nod position for a predetermined amount of time, e.g., five seconds, and then relax. The user may be instructed to gently move their head back to the normal start position.

FIGS. 2C-2D illustrate a head rotation exercise that may be utilized in various embodiments of the systems described herein. The user may be instructed to gently turn their head from one side to the other. The user may be instructed to progressively aim to turn their head far enough so their chin is in line with their shoulder and they can see the wall in line with their shoulder. The user may be instructed to repeat any of the above exercises up to a predetermined number of times, e.g., ten times, per side.

FIGS. 2E-2F illustrate a shoulder blade exercise that may be utilized in various embodiments of the systems described herein. The user may be instructed to lie on their right side with their arm resting up on two pillows. The user may be instructed to roll their left shoulder blade back and across their ribs towards the center of their back and hold this position for a predetermined amount of time, e.g., ten seconds. The user may be instructed to repeat this exercise up to a predetermined number of times, e.g., five times and to also repeat the exercise while lying on the left side for the right shoulder blade.

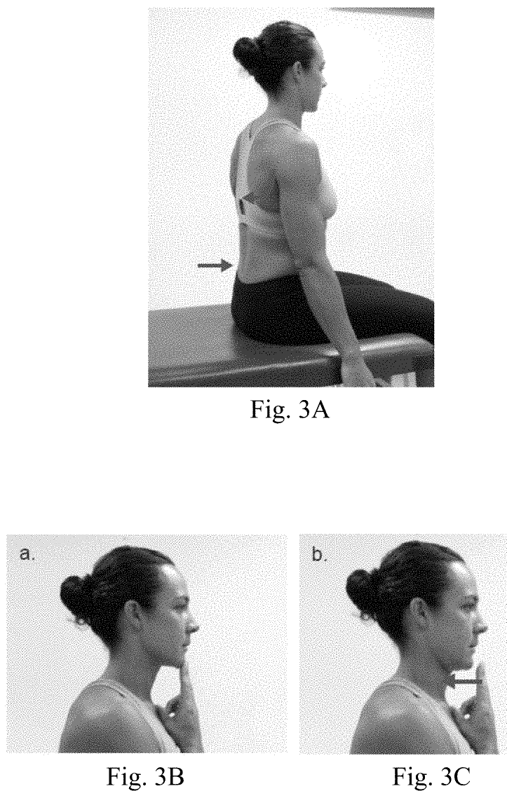

FIGS. 3A-3C illustrate exemplary user motion according to embodiments of the present disclosure. In particular, FIGS. 3A-3C illustrate a correct postural position (FIG. 3A) and a neck exercise (FIGS. 3B-3C) performed while sitting that may be utilized in various embodiments of the systems described herein. In various embodiments, the user may be instructed to assume a particular posture before and/or during the exercise. The user may be instructed to correct their posture regularly by gently straightening up their lower back and pelvis (to sit tall). The user may be instructed to gently draw back their shoulder blades back and down. The user may be instructed to gently tuck in their chin and to hold the position with ease for a predetermined amount of time, e.g., ten seconds. The user may be instructed that this position will prevent and ease muscle pain and tension in their neck and shoulder muscles. The user may be instructed to repeat the correction regularly, e.g., every half hour during the day.

FIGS. 3B-3C illustrate a neck retraction exercise that may be utilized in various embodiments of the systems described herein. The user may be instructed to sit in the correct position described and illustrated in FIG. 3A. The user may be instructed to gently draw their head back, sliding their chin back horizontally and keeping their nose pointing straight ahead. The user may be instructed that they should feel the retraction movement at the base of their neck and their neck should stay long. The user may be instructed to repeat this a predetermined number of times, e.g., ten times every hour while sitting.

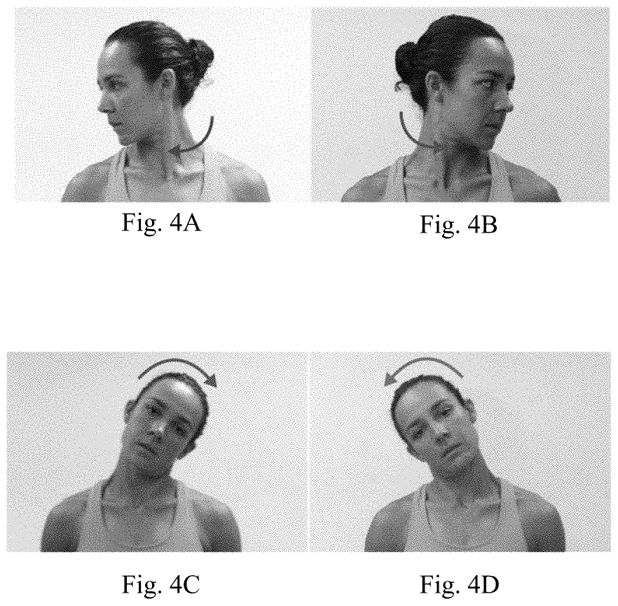

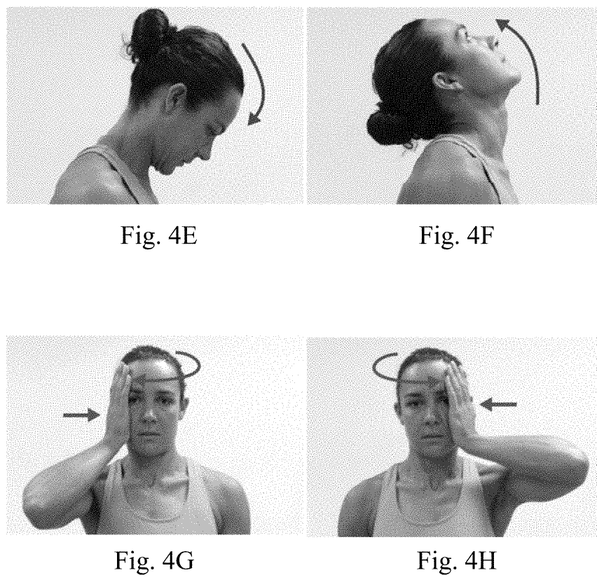

FIGS. 4A-4H illustrate exemplary user motion according to embodiments of the present disclosure. In particular, FIGS. 4A-4H illustrate various neck movement exercises that may be utilized in various embodiments of the systems described herein. The user may be instructed to sit in the correct position described and illustrated in FIG. 3A before performing any of the below exercises.

FIGS. 4A-4B illustrate neck rotation where the user may be instructed to gently turn their head from one side to the other. The user may be instructed to progressively aim their head so that they see the wall in line with their shoulder.

FIGS. 4C-4D illustrate neck side bending where the user may be instructed to gently tilt their head towards their shoulder and feel the gentle stretch in the muscles on the side of the neck. The user may be instructed to perform the movement to both sides.

FIGS. 4E-4F illustrate neck bending and extension where the user may be instructed to gently bend their head towards their chest. The user may be instructed to lead the movement with their chin and, moving the chin first, to bring their head back to the upright position and gently roll it back to look up towards the ceiling. The user may be instructed to, leading with their chin, return their head to the upright position. Any of the above exercises may be performed a predetermined number of times, e.g., ten times.

FIGS. 4G-4H illustrate various neck strengthening exercises where the user may be instructed to make sure their chin is relaxed and slightly down. The user may be instructed to place their right hand on their right cheek. The user may be instructed to gently try to turn their head into their fingers to look over their right shoulder but allow no movement. The user may be instructed to hold the contraction for a predetermined amount of time, e.g., five seconds. The user may be instructed to use a 10% to 20% effort and no more. The user may be instructed to repeat this exercise with the left hand on the left cheek. The user may be instructed to do five repetitions of the holding exercise to each side.

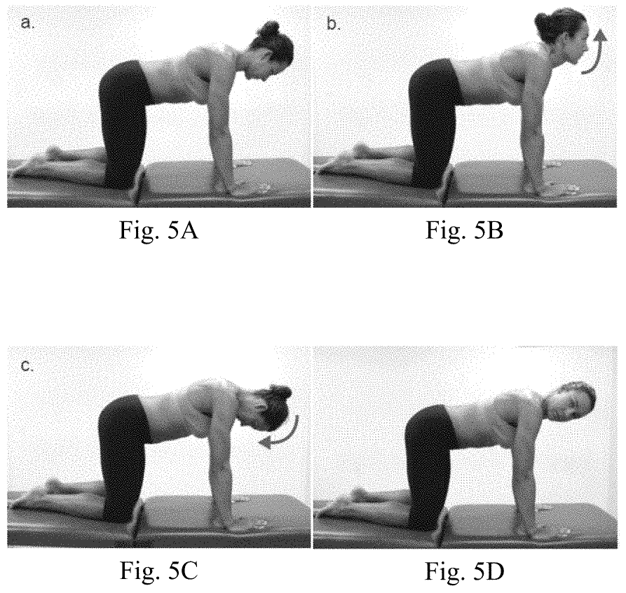

FIGS. 5A-5D illustrate exemplary user motion according to embodiments of the present disclosure. In particular, FIGS. 5A-5D illustrate various neck strengthening exercises performed in a four-point kneeling position that may be utilized in various embodiments of the systems described herein.

The user may be instructed to first adopt a four-point kneeling position (FIG. 5A) before performing any of the below exercises. To adopt the four-point kneeling position as shown in FIG. 5A, the user may be instructed to begin by ensuring their knees are directly under their hips, and their hands directly under their shoulders. The user may be instructed that their lower back should be in a neutral position; that is, with a natural arch. The user may be instructed to gently draw their belly button to their spine (10% effort). The user may be instructed to push gently through their shoulder blades, so that their upper back is level. The user may be instructed to draw their shoulders gently away from their ears, or toward their hips. The user may be instructed to lift their head up so that it is level with their shoulders, but maintain a gentle chin tucked or nod position.

FIGS. 5B-5C illustrate neck bending and extension in the four-point kneeling position. The user may be instructed to slowly look up toward the ceiling as far as they can go. The user may be instructed to hold this position for 5 to 10 seconds. The user may be instructed to slowly bend their neck, leading the movement with a chin tuck or nodding action. The user may be instructed to continue the neck bending movement as far as possible. The user may be instructed to aim for their chin to touch their chest. The user may be instructed that throughout this movement they should hold the neutral lower back and shoulder blade posture described above. The user may be instructed to perform this exercise a predetermined number of times, e.g., five to ten times.

FIG. 5D illustrates neck bending in the four-point kneeling position. The user may be instructed to slowly rotate their head (turn their neck to one side). The user may be instructed that it is important to maintain the gentle chin tuck or ‘nod’ position throughout the movement. The user may be instructed to make sure their head stays level with their body, and does not drop down. The user may be instructed that, if they do this exercise correctly, they should be looking over their shoulder at the end of the movement. The user may be instructed that it helps to do this exercise positioning themselves side-on to a mirror so that they can check their head position. The user may be instructed to repeat this exercise to the other side. The user may be instructed to perform this exercise a predetermined number of times, e.g., five to ten times.

In various embodiments, training protocols are based on standard rehabilitation exercises. For example, additional neck movements suitable for neck rehabilitation using various embodiments of the systems described herein may be found in Guidelines for the management of acute whiplash associated disorders for health professionals, 3rd Edition, 2014, available at https://www.sira.nsw.gov.au/resources-library/motor-accident-resources/publications/for-professionals/whiplash-resources/SIRA08104-Whiplash-Guidelines-1117-396479.pdf, which is hereby incorporated by reference. However, it will be appreciated that the versatility of the virtual environment enables a range of exercises that are not practical when relying on physical cues.

In an exemplary neck physical training protocol, a 2D or 3D object moves in the space around the user. The user is directed to follow the object with their gaze, thus moving their neck in the direction the object moves, performing the neck movements suitable for neck rehabilitation.

In an exemplary arm/shoulder/back rehabilitation protocol, a 2D or 3D object moves in the space around the user. The user is directed to follow the object with their arm position, thus moving their arm in the direction the object moves.

It will be appreciated that this process can be applied to various physical rehabilitation protocols for any body part (e.g., the neck, arm, leg, back, hip, elbow, wrist, ankle, or fingers).

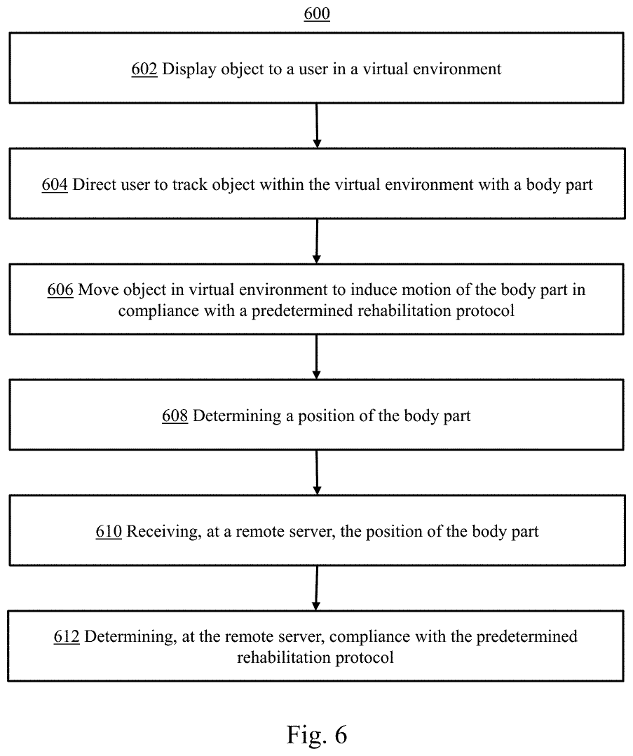

Referring to FIG. 6, a method 600 of guiding user motion according to embodiments of the present disclosure is illustrated. At 602, an object is displayed to a user within a virtual environment. At 604, the user is directed to track the object within the virtual environment with a body part. At 606, the object is moved within the virtual environment to induce motion of the body part in compliance with a predetermined rehabilitation protocol. At 608, a position is determined of the body part. At 610, the position of the body part is received at a remote server. At 612, compliance with the predetermined rehabilitation protocol is determined at the remote server.

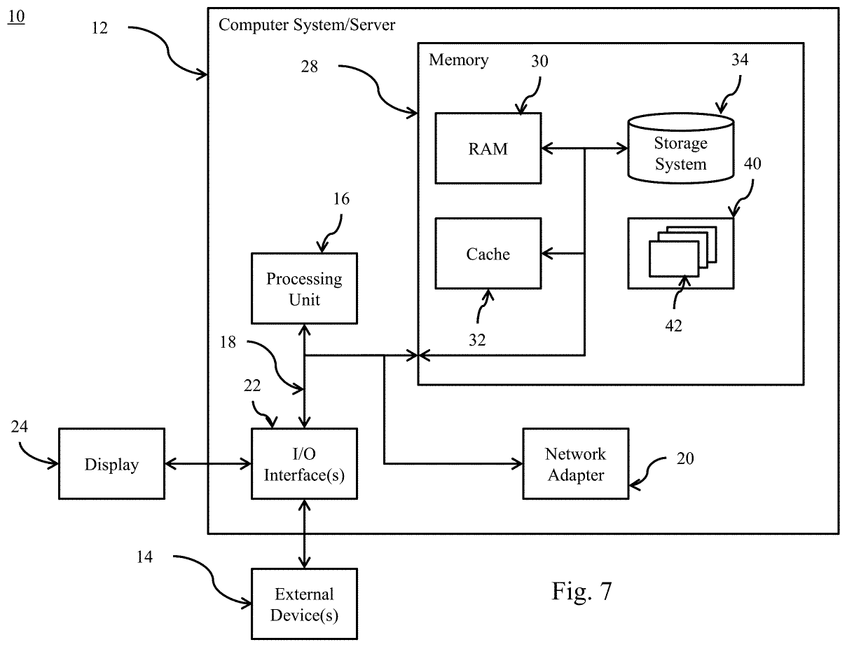

Referring now to FIG. 7, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 7, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

A Picture Archiving and Communication System (PACS) is a medical imaging system that provides storage and access to images from multiple modalities. In many healthcare environments, electronic images and reports are transmitted digitally via PACS, thus eliminating the need to manually file, retrieve, or transport film jackets. A standard format for PACS image storage and transfer is DICOM (Digital Imaging and Communications in Medicine). Non-image data, such as scanned documents, may be incorporated using various standard formats such as PDF (Portable Document Format) encapsulated in DICOM.

An electronic health record (EHR), or electronic medical record (EMR), may refer to the systematized collection of patient and population electronically-stored health information in a digital format. These records can be shared across different health care settings and may extend beyond the information available in a PACS discussed above. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Health Level-7 or HL7 refers to a set of international standards for transfer of clinical and administrative data between software applications used by various healthcare providers. These standards focus on the application layer, which is layer 7 in the OSI model. Hospitals and other healthcare provider organizations may have many different computer systems used for everything from billing records to patient tracking. Ideally, all of these systems may communicate with each other when they receive new information or when they wish to retrieve information, but adoption of such approaches is not widespread. These data standards are meant to allow healthcare organizations to easily share clinical information. This ability to exchange information may help to minimize variability in medical care and the tendency for medical care to be geographically isolated.

In various systems, connections between a PACS, Electronic Medical Record (EMR), Hospital Information System (HIS), Radiology Information System (RIS), or report repository are provided. In this way, records and reports form the EMR may be ingested for analysis. For example, in addition to ingesting and storing HL7 orders and results messages, ADT messages may be used, or an EMR, RIS, or report repository may be queried directly via product specific mechanisms. Such mechanisms include Fast Health Interoperability Resources (FHIR) for relevant clinical information. Clinical data may also be obtained via receipt of various HL7 CDA documents such as a Continuity of Care Document (CCD). Various additional proprietary or site-customized query methods may also be employed in addition to the standard methods.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method comprising:
displaying an object to a user within a virtual environment;
directing the user to track the object within the virtual environment with a body part;
moving the object within the virtual environment to induce motion of the body part in compliance with a predetermined rehabilitation protocol;
determining a plurality of positions of the body part;
receiving, at a remote server, the position of the body part; and
determining, at the remote server, compliance with the predetermined rehabilitation protocol.

2. (canceled)

3. The method of claim 1, wherein determining compliance comprises:
comparing the plurality of positions of the body part with a plurality of predetermined positions;
determining a compliance factor based on the comparing; and
determining whether the compliance factor is above a predetermined threshold.

4. The method of claim 3, wherein the plurality of predetermined positions represent positions along a three-dimensional path corresponding to the rehabilitation protocol.

5. The method of claim 3, wherein comparing comprises determining a difference between the plurality of positions of the body part and the plurality of predetermined positions.

6. (canceled)

7. The method of claim 1, wherein determining compliance comprises determining compliance with an electronic health record.

8. (canceled)

9. (canceled)

10. (canceled)

11. The method of claim 1, further comprising directing the user to assume a predetermined posture before directing the user to track the object.

12. The method of claim 11, further comprising determining whether the user has assumed the predetermined posture.

13. (canceled)

14. (canceled)

15. A system comprising:
a virtual reality display adapted to display a virtual environment to a user;
a computing node comprising a computer readable storage medium having program instructions embodied therewith, the program instructions executable by a processor of the computing node to cause the processor to perform a method comprising:
displaying an object to the user within a virtual environment via the virtual reality display;
directing the user to track the object within the virtual environment with a body part;
moving the object within the virtual environment to induce motion of the body part in compliance with a predetermined rehabilitation protocol;
determining a plurality of positions of the body part;
receiving, at a remote server, the position of the body part; and
determining, at the remote server, compliance with the predetermined rehabilitation protocol.

16. (canceled)

17. The system of claim 15, wherein determining compliance comprises:
comparing the plurality of positions of the body part with a plurality of predetermined positions;
determining a compliance factor based on the comparing; and
determining whether the compliance factor is above a predetermined threshold.

18. The system of claim 17, wherein the plurality of predetermined positions represent positions along a three-dimensional path corresponding to the rehabilitation protocol.

19. The system of claim 17, wherein comparing comprises determining a difference between the plurality of positions of the body part and the plurality of predetermined positions.

20. (canceled)

21. The system of claim 15, wherein determining compliance comprises determining compliance with an electronic health record.

22. (canceled)

23. (canceled)

24. (canceled)

25. The system of claim 15, the program instructions further executable by the processor to perform the method comprising directing the user to assume a predetermined posture before directing the user to track the object.

26. (canceled)

27. (canceled)

28. (canceled)

29. A computer program product for guiding user motion, the computer program product comprising a computer readable storage medium having program instructions embodied therewith, the program instructions executable by a processor to cause the processor to perform a method comprising:
displaying an object to a user within a virtual environment;
directing the user to track the object within the virtual environment with a body part;
moving the object within the virtual environment to induce motion of the body part in compliance with a predetermined rehabilitation protocol;
determining a plurality of positions of the body part;
receiving, at a remote server, the position of the body part; and
determining, at the remote server, compliance with the predetermined rehabilitation protocol.

30. (canceled)

31. The computer program product of claim 29, wherein determining compliance comprises:
comparing the plurality of positions of the body part with a plurality of predetermined positions;
determining a compliance factor based on the comparing; and
determining whether the compliance factor is above a predetermined threshold.

32. The computer program product of claim 31, wherein the plurality of predetermined positions represent positions along a three-dimensional path corresponding to the rehabilitation protocol.

33. The computer program product of claim 31, wherein comparing comprises determining a difference between the plurality of positions of the body part and the plurality of predetermined positions.

34. (canceled)

35. The computer program product of claim 29, wherein determining compliance comprises determining compliance with an electronic health record.

36. (canceled)

37. (canceled)

38. (canceled)

39. The computer program product of claim 29, the program instructions further executable by the processor to perform the method comprising directing the user to assume a predetermined posture before directing the user to track the object.

40. The computer program product of claim 39, the program instructions further executable by the processor to perform the method comprising determining whether the user has assumed the predetermined posture.

41. (canceled)

42. (canceled)

43. (canceled)
